SEQUENCE LISTING (38 sequences)

SEQ ID NO: 1 - DNA, 2013 bp, Arabidopsis thaliana ecotype Columbia
atggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtacatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatactga

SEQ ID NO: 2 - DNA, 2013 bp, Arabidopsis thaliana ecotype Columbia
atggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgtgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatactga

SEQ ID NO: 3 - protein, 670 aa, Arabidopsis thaliana ecotype Columbia
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPRKGADILVEALERQGVETVFAYPGGTSMEIHQALTRSSSIRNVLPRHEQGGVFAAEGYARSSGKPGICIATSGPGATNLVSGLADALLDSVPLVAITGQVPRRMIGTDAFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVENLPVKVLLLNNQHLGMVMQWEDRFYKANRAHTFLGDPAQEDEIFPNMLLFAAACGIPAARVTKKADLREAIQTMLDTPGPYLLDVICPHQEHVLPMIPSGGTFNDVITEGDGRIKY

SEQ ID NO: 4 - protein, 670 aa, Arabidopsis thaliana ecotype Columbia
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPRKGADILVEALERQGVETVFAYPGGASMEIHQALTRSSSIRNVLPRHEQGGVFAAEGYARSSGKPGICIATSGPGATNLVSGLADALLDSVPLVAITGQVPRRMIGTDVFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVENLPVKVLLLNNQHLGMVMQWEDRFYKANRAHTFLGDPAQEDEIFPNMLLFAAACGIPAARVTKKADLREAIQTMLDTPGPYLLDVICPHQEHVLPMIPSGGTFNDVITEGDGRIKY

SEQ ID NO: 5 - DNA, 38 bp, Arabidopsis thaliana
tgtaaaacgacggccagtcttgtatccattctcttaac

SEQ ID NO: 6 - DNA, 38 bp, Arabidopsis thaliana
caggaaacagctatgaccggcggagatggaggagggag

SEQ ID NO: 7 - DNA, 38 bp, Arabidopsis thaliana
tgtaaaacgacggccagttcacaagtctcttcttcttc

SEQ ID NO: 8 - DNA, 38 bp, Arabidopsis thaliana
caggaaacagctatgacccacctccttgttcgtgacga

SEQ ID NO: 9 - DNA, 38 bp, Arabidopsis thaliana
tgtaaaacgacggccagtgtgctcaacacaaccaccaa

SEQ ID NO: 10 - DNA, 38 bp, Arabidopsis thaliana
caggaaacagctatgaccatatcttcaacatccatcac

SEQ ID NO: 11 - DNA, 38 bp, Arabidopsis thaliana
tgtaaaacgacggccagttattcgcagcagaaggatac

SEQ ID NO: 12 - DNA, 38 bp, Arabidopsis thaliana
caggaaacagctatgaccgctagaattcaaacaaccac

SEQ ID NO: 13 - DNA, 38 bp, Arabidopsis thaliana
tgtaaaacgacggccagtccctaggattattgaggaag

SEQ ID NO: 14 - DNA, 38 bp, Arabidopsis thaliana
caggaaacagctatgaccagtcaatatcaatatgaaca

SEQ ID NO: 15 - DNA, 38 bp, Arabidopsis thaliana
tgtaaaacgacggccagtgatgaattgggtaggtttgt

SEQ ID NO: 16 - DNA, 38 bp, Arabidopsis thaliana
caggaaacagctatgaccattatggcttttccatcagt

SEQ ID NO: 17 - DNA, 38 bp, Arabidopsis thaliana
tgtaaaacgacggccagtcggctgagattgggaagaat

SEQ ID NO: 18 - DNA, 38 bp, Arabidopsis thaliana
caggaaacagctatgaccgtttaataaaagtaccttca

SEQ ID NO: 19 - DNA, 38 bp, Arabidopsis thaliana
tgtaaaacgacggccagtaagtactggtgtcgggcaac

SEQ ID NO: 20 - DNA, 38 bp, Arabidopsis thaliana
caggaaacagctatgaccacacatgttcttggtgcgga

SEQ ID NO: 21 - DNA, 38 bp, Arabidopsis thaliana
tgtaaaacgacggccagtaaccagcatcttggcatggt

SEQ ID NO: 22 - DNA, 38 bp, Arabidopsis thaliana
caggaaacagctatgacctgaaagaaaggaaaccaaac

SEQ ID NO: 23 - DNA, 50 bp, Arabidopsis thaliana
gggcccgcggccgcagtctcatttttaaacaaatcatgttcacaagtctc

SEQ ID NO: 24 - DNA, 44 bp, Arabidopsis thaliana
gggcccctgcagtctctcagtatttaatccggccatctccttcc

SEQ ID NO: 25 - DNA, 2013 bp, Arabidopsis thaliana ecotype Landsberg erecta
atggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgtgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggacggtgtatgcgaattacgctgtggagcatagtgatttgttgttggcgtttggggtgaggtttgatgatcgcgtcacgggtaagcttgaggcttttgctagtagggctaagattgttcatattgatattgactctgctgagattgggaagaataagactcctcatgtgtctgtgtgtggtgatgtcaagctggctttgcaagggatgaataaggttcttgagaaccgagctgaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccattataagtactggtgtcgggcaacatcaaatgtgggcggcgcagttctacaattacaagaagccaaggcagtggctatcatcaggaggccttggagctatgggttttggacttcctgctgccattggagcgtctgttgctaaccctgatgcaatagttgtggatattgacggagatggaagctttataatgaatgtgcaagagctggccacaatccgtgtagagcaacttccagtgaagatactcttattaaacaaccagcatcttggcatggttatgcaatgggaagatcggttctacaaggctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatactga

SEQ ID NO: 26 - protein, 670 aa, Arabidopsis thaliana ecotype Landsberg erecta
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPRKGADILVEALERQGVETVFAYPGGASMEIHQALTRSSSIRNVLPRHEQGGVFAAEGYARSSGKPGICIATSGPGATNLVSGLADALLDSVPLVAITGQVPRRMIGTDVFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVEQLPVKILLLNNQHLGMVMQWEDRFYKANRAHTFLGDPAQEDEIFPNMLLFAAACGIPAARVTKKADLREAIQTMLDTPGPYLLDVICPHQEHVLPMIPSGGTFNDVITEGDGRIKY

SEQ ID NO: 27 - protein, 31 aa, Arabidopsis thaliana
LERQGVETVFAYPGGASMEIHQALTRSSSIR

SEQ ID NO: 28 - protein, 31 aa, Brassica napus
LERQGVETVFAYPGGASMEIHQALTRSSTIR

SEQ ID NO: 29 - protein, 31 aa, Gossypium hirsutum
LEREGVKDVFAYPGGASMEIHQALTRSKIIR

SEQ ID NO: 30 - protein, 31 aa, Nicotiana tabacum; identical to SEQ ID NO: 29

SEQ ID NO: 31 - protein, 31 aa, Glycine max
LERQGVTDVFAYPGGASMEIHQALTRSSSIR

SEQ ID NO: 32 - protein, 31 aa, Zea mays
LERCGVRDVFAYPGGASMEIHQALTRSPVIA

SEQ ID NO: 33 - protein, 31 aa, Arabidopsis thaliana
VAITGQVPRRMIGTDAFQETPIVEVTRSITK

SEQ ID NO: 34 - protein, 31 aa, Brassica napus; identical to SEQ ID NO: 33

SEQ ID NO: 35 - protein, 31 aa, Gossypium hirsutum; identical to SEQ ID NO: 33

SEQ ID NO: 36 - protein, 31 aa, Nicotiana tabacum; identical to SEQ ID NO: 33

SEQ ID NO: 37 - protein, 31 aa, Glycine max; identical to SEQ ID NO: 33

SEQ ID NO: 38 - protein, 31 aa, Zea mays; identical to SEQ ID NO: 33

(19) United States
(12) Patent Application Publication
(10) Pub. No.: US 2003/0097692 A1
(43) Pub. Date: May 22, 2003

(54) Plants with imidazolinone-resistant ALS

(76) Inventors: Georg Jander, Jamaica Plain, MA (US); Timothy P. Durrett, Clayton, MO (US); Scott R. Baerson, Oxford, MI (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/004,827

(22) Filed: Dec. 7, 2001

Related U.S. Application Data
(60) Provisional application No. 60/257,480, filed on Dec. 21, 2000.

(51) Int. Cl. C12N 15/82; A01H 5/00; C12N 5/10; C12N 15/29; C12N 5/04

(52) U.S. Cl. 800/300; 800/278; 435/418; 435/419; 435/6; 536/23.6

(57) ABSTRACT

Acetolactate synthase (ALS), a key enzyme in the biosynthesis of valine, leucine and isoleucine in plants is inhibited by herbicides comprising imidazolinones. The present invention relates to Arabidopsis thaliana genes encoding a mutant acetolactate synthase (ALS) enzyme that is specifically resistant to imidazolinone herbicides. Exemplary of these genes are DNA sequences which encode an amino acid substitution at position 122 or an amino acid substitution at position 205 of the wild-type ALS enzyme in Arabidopsis thaliana, ecotype Columbia or an amino acid substitution at position 205 of the wild-type ALS enzyme in Arabidopsis thaliana, ecotype Landsberg erecta. The mutant ALS genes can be used to transform plants to herbicide resistance; in this regard, the invention also provides host cells and vectors containing the gene, which cells and vectors are useful in the transformation process. The mutant ALS genes is commercially useful, when used to impart imidazolinone resistance to a crop plant; thereby permitting the utilization of the imidazolinone or analogous herbicide as a single application at a concentration which ensures the complete or substantially complete killing of weeds, while leaving the transgenic crop plant essentially undamaged.

### CROSS-REFERENCE TO RELATED APPLICATION

This application claims priority under 35 U.S.C. §19(e) of U.S. Provisional Application No. 60/257,480 filed Dec. 21, 2000, the disclosure of which application is incorporated herein by reference in its entirety.

### FIELD OF THE INVENTION

Disclosed herein are DNA sequences that encode variant forms of acetolactate synthase (ALS; also known as acetohydroxyacid synthase or AHAS), which is an essential enzyme routinely produced in a variety of plants and a broad range of microorganisms. The function of wild type ALS is inhibited by imidazolinone herbicides; however, novel ALS variants function in the presence of imidazolinone herbicides and, therefore can be used to confer herbicide resistance to plants or other organisms containing them.

### BACKGROUND OF THE INVENTION

The use of herbicides in agriculture is now widespread. Although there are a large number of available compounds which effectively destroy weeds, not all herbicides are capable of selectively targeting the undesirable plants over crop plants. Often, it is necessary to settle for compounds which are simply less toxic to crop plants than to weeds. In order to overcome this problem, development of herbicide resistant crop plants has become a major focus of agricultural research.

An important aspect of development of herbicide-resistance is an understanding of the herbicide target, and then manipulating the affected biochemical pathway in the crop plant so that the inhibitory effect is avoided while the plant retains normal biological function. One of the first discoveries of the biochemical mechanism of herbicides related to a series of structurally unrelated herbicide compounds, the imidazolinones, the sulfonylureas and the triazolopyrimidines. It is now known (Shaner et al. Plant Physiol. 76: 545-546,1984; U.S. Pat. No. 4,761,373) that each of these herbicides inhibits plant growth by interference with ALS—an essential enzyme required for plant growth, e.g. in the synthesis of the amino acids isoleucine, leucine and valine.

In tobacco, ALS function is reported to be encoded by two unlinked genes, SURA and SURB. There is substantial identity between the two genes, both at the nucleotide level and amino acid level in the mature protein, although the N-terminal, putative transit region differs more substantially (Lee et al, EMBO J. 7: 1241-1248, 1988). Arabidopsis, on the other hand, is reported to have a single ALS gene (Mazur et al., Plant Physiol. 85:1110-1117, 1987). U.S. Pat. Nos. 5,013,659 and 5,605,011 (incorporated herein by reference) disclose comparisons among sequences of ALS genes in higher plants i.e. tobacco, Arabidopsis thaliana, sugar beet and corn showing a high level of conservation of certain regions of the sequence. Specifically, there are at least 10 regions of amino acid sequence conservation among yeast, E. coli, Arabidopsis thaliana and tobacco with reference to FIG. 6 of U.S. Pat. No. 5,605,011, the amino acids are conserved at positions 121, 122, 197, 205, 256, 359, 384, 588, 591, and 595. U.S. Pat. No. 5,013,659 further reports that mutants exhibiting herbicide resistance have an amino acid substitution in one or more of these conserved regions. In particular, substitution of certain amino acids for the wild type amino acid at these specific sites in the ALS protein sequence have been shown to be tolerated, and indeed result in herbicide resistance of the plant possessing this mutation, while retaining catalytic function.

Reference is made to U.S. Pat. No. 5,605,011 which discloses amino acid substitutions to confer herbicide resistance. The patent discloses amino acid substitutions in yeast for alanine at position 122 resulting in sulfonylurea-resistant ALS in yeast. However, alanine to proline substitution in a tobacco SURB ALS did not yield chlorsulfuron resistance when expressed in sugar beet transformants. Yeast with amino acid substitutions for alanine at position 205, e.g. with cysteine, glutamic acid, arginine, tryptophan, tyrosine, valine or asparagine result in sulfonylurea-resistant ALS. Although general resistance to the group of herbicides comprising sulfonylureas, trizolopyrimidines and imidazolinones is reported, only resistance to sulfonylureas was demonstrated.

Early herbicide-enzyme kinetics data by Schloss et al., in Nature 331:360-362 (1988), proposed that sulfonylureas, imidazolinones and trizolopyrimidines shared a common binding site on a bacterial ALS. However, additional studies by several inventors, including experiments by Saxena et al., Plant Physiol. 94:1111-1115 (1990) and Sathasivan et al., Nucleic Acids Res. 18:2188 (1990), have indicated that with the exception of a few cases, the mutant forms of ALS which were resistant to imidazolinone lacked cross-resistance to sulfonylureas. Therefore, identification of the mutation site(s) in the ALS gene which code for the mutant plant's imidazolinone resistance is of agricultural significance.

Furthermore, the mechanism of inhibition was shown to be dissimilar between the imidazolinone and sulfonylurea herbicides. Imidazolinones inhibit ALS activity by binding-noncompetitively to a common site on the enzyme, as demonstrated by Shaner et al., Plant Physiol. 76:545-546 (1984). By comparison, sulfonylureas inhibit ALS activity by competition as described by La Rossa and Schloss in J. Biol. Chem. 259:8753-8757 (1984). Therefore, since the mechanism of action of imidazolinone appears to be different from that of sulfonylurea herbicides, understanding the molecular basis of imidazolinone resistance is of great interest.

Imidazolinone-specific resistance has been reported elsewhere in a number of plants. U.S. Pat. No. 4,761,373 generally described an altered ALS as a basis of herbicide resistance in plants, and specifically disclosed certain imidazolinone resistant corn lines.

U.S. Pat. No. 5,731,180 discloses a corn AHAS mutant (i.e. an ALS) with an amino acid substitution at position 621 which causes imidazolinone-specific resistance.

Haughn et al. (Mol. Gen. Genet. 211:266-271, 1988) disclosed the occurrence of imidazolinone resistance in Arabidopsis. Sathasivan et al. (U.S. Pat. No. 5,767,366) identified the imidazolinone-specific resistance in Arabidopsis as being based on a mutation at position 653 in the normal ALS sequence.

### SUMMARY OF THE INVENTION

The present invention provides nucleic acid molecules which encode a functional acetolactate synthase (ALS) which has (a) an alanine-to-threonine substitution at amino acid sequence position 122, or (b) an alanine-to-valine substitution at amino acid sequence position 205, relative to the amino acid sequence alignment of FIGS. 1 and 2.

In one embodiment, a host cell's DNA is mutated to encode (a) an alanine-to-threonine substitution at amino acid sequence position 122, or (b) an alanine-to-valine substitution at amino acid sequence position 205, relative to the amino acid sequence alignment of FIGS. 1 and 2.

In another embodiment, the present invention provides nucleic acid fragments encoding imidazolinone-resistant ALS which may be incorporated into a nucleic acid construct used to transform a host cell, preferably a plant, more preferably a plant selected from the group consisting of Arabidopsis thaliana, maize, soybean, wheat, cotton, canola, rice and sunflower, containing wild type ALS.

The present invention also provides transformed plants exhibiting imidazolinone resistance having a nucleic acid molecule which comprises: (a) an exogenous promoter region which functions in a plant cell to cause the production of a mRNA molecule; (b) a structural nucleic acid molecule encoding functional ALS comprising an amino acid sequence of SEQ ID NO: 3 or SEQ ID NO: 4 or a homolog thereof having an alanine-to-threonine substitution at position 122 or an alanine-to-valine substitution at position 205, and (c) a 3′ non-translated sequence that functions in the plant cell to cause termination of transcription and addition of polyadenylated ribonucleotides to a 3′ end of the mRNA molecule.

In a preferred embodiment, the transformed plant of this invention is rice, cotton, wheat, canola, maize, soybean, sunflower and Arabidopsis thaliana.

The present invention further provides a method of conferring imidazolinone-specific resistance to a plant cell by providing the plant cell with a nucleic acid sequence encoding functional ALS having either (a) an alanine-to-threonine substitution at amino acid sequence position 122, or (b) an alanine-to-valine substitution at amino acid sequence position 205, relative to the amino acid sequence alignment of FIGS. 1 and 2.

The present invention also provides novel selectable markers for use in transformation experiments. In one embodiment of the invention, nucleic acid constructs comprising the mutant ALS nucleic acid sequence is linked to a gene encoding an agronomically useful trait.

The present invention provides a method using imidazolinone resistance as a selectable marker in a cell or organism wherein said resistance is provided by nucleic acid sequence encoding functional ALS having an alanine-to-threonine substitution at position 122 or an alanine-to-valine substitution at position 205 relative to the amino acid sequence of FIG. 1 or 2.

The present invention provides methods of conferring imidazolinone resistance to plants, methods for determining the imidazolinone tolerance of plants, and methods for introgressing an agronomically useful trait into plants using nucleic acid molecules of this invention.

### IN THE DRAWINGS

FIGS. 1 and 2 are amino acid alignments of peptide segments of crop plants around Arabidopsis thaliana ALS amino acid positions.

### DETAILED DESCRIPTION OF THE INVENTION

The present invention provides novel DNA sequences derived from Arabidopsis thaliana ecotype Columbia and Arabidopsis thaliana ecotype Landsberg erecta and have a substitution of an amino acid at position 122 or at position 205 of the normal ALS sequence. This substitution in the ALS gene sequence results in a functional enzyme, but renders the enzyme specifically resistant to inhibition by imidazolinone herbicides. The availability of these variant sequences provides a tool for transformation of different crop plants to imidazolinone herbicide resistance, as well as providing novel selectable markers for use in other types of genetic transformation experiments.

The following definitions should be understood to apply throughout the specification and claims.

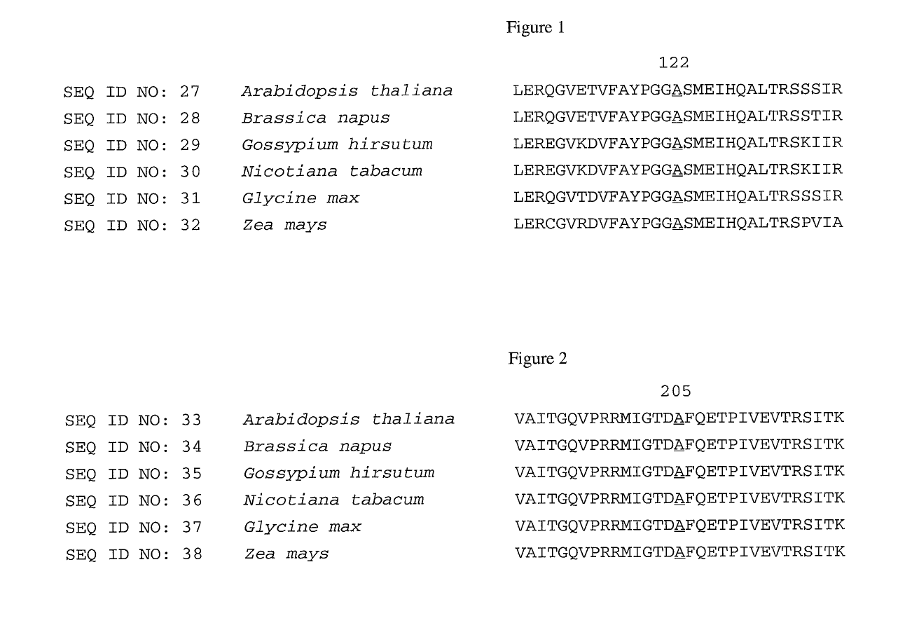

“Functional” or “normal” ALS is an enzyme which is capable of catalyzing a step in the pathway for synthesis of the essential amino acids isoleucine, leucine and valine. A “wild-type” ALS is an imidazolinone sensitive enzyme. Wild-type ALS amino acid sequence has alanine at amino acid sequence positions 122 and 205 with reference to Arabidopsis thaliana ALS and FIGS. 1 and 2.

A “resistant” plant is one which produces a mutant but functional ALS enzyme, and which is capable of reaching maturity when grown in the presence of normally inhibitory levels of imidazolinone, e.g. at least an I₁₀₀ dose, more preferably up to about 2×I₁₀₀ dose or higher. The term “resistant”, as used herein, is also intended to encompass “tolerant” plants, i.e., those plants which phenotypically evidence adverse, but not lethal, reactions to the imidazolinone in a dose which is lethal to wild type. As used herein, “imidazolinone-specific resistance” means resistance to an imidazolinone herbicide but not to a sulfonylurea herbicide.

As used herein, the group “imidazolinones” is meant to encompass a class of herbicides. The imidazolinone herbicides, notably imazapyr, imazaquin and imazethapyr, are a particularly important class of herbicide As described in the “Herbicide Handbook of the Weed Science Society of America”, 6th Ed., (1989), imazapyr (2-[4,5-dihydro-4-methyl-4-(1-methylethyl)-5-oxo-1H-imidazol-2-yl]-3-pyridinecarboxylic acid), is a non-specific, broad-spectrum herbicide, whereas both imazaquin (2-[4,5-dihydro-4-methyl-4-(1-methylethyl)-5-oxo-1H-imidazol-2-yl]-3-quinolinecarboxylic acid), and imazethapyr (2-[4,5-dihydro-4-methyl-4-(1-methylethyl)-5-oxo-1H-imidazol-2-yl]-5-ethyl -3-pyridinecarboxylic acid) are crop-specific herbicides particularly suited to use with soybean or peanut crops. These herbicides offer low mammalian toxicity, permit low application rates to plant crops, and provide long duration broad-spectrum weed control in the treatment of agricultural crops. A crop made more resistant to imidazolinone herbicides offers a selective means to control and kill weeds without adversely affecting the crop plant.

The agents of the present invention will preferably be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic and thus involve the capacity of the agent to mediate a chemical reaction or response.

The agents of the present invention may also be “recombinant”. As used herein, the term recombinant describes (a) nucleic acid molecules that are constructed or modified outside of cells and that can replicate or function in a living cell, (b) molecules that result from the transcription, replication or translation of recombinant nucleic acid molecules, or (c) organisms that contain recombinant nucleic acid molecules or are modified using recombinant nucleic acid molecules.

(a) Nucleic Acid Molecules

This invention provides mutant ALS genes, which have been isolated from imidazolinone-resistant plants, and homologs thereof. This invention further provides the nucleic acid sequence of these genes and the amino acid sequence of functional ALS encoded by these genes. This invention also provides nucleic acid molecules and constructs and methods for transforming an imidazolinone-sensitive plant to confer greater imidazolinone resistance than that originally possessed by the transformed plant.

The ALS genes of this invention may be isolated and/or purified from a higher plant, particularly a plant shown capable of resisting imidazolinone treatment. The plant can be the mutant result of various mutagenic processes, including chemical, biological, radioactive, or ultraviolet treatments. Alternatively, the imidazolinone-resistant plant can be the result of growing selected plants in soil or other medium at increasingly higher concentrations of imidazolinone until the plants which survive have developed imidazolinone-resistant ALS enzymes. Regardless of the source of the imidazolinone-resistant organism, screening must show that the ALS gene therein effectively codes for an imidazolinone-resistant ALS enzyme.

Once one or more host strains have been identified, any of a variety of commonly used techniques may be employed to identify the coding sequence for the imidazolinone-resistant ALS, e.g. to isolate the desired DNA fragment and clone it into a vector where it may be transformed into a host to characterize its expression. Those skilled in the art know how to isolate a homolog by making a library, and screening it for homology to a gene or protein of interest. Methods of isolating mRNA and making cDNA are also known to those skilled artisans.

One skilled in the art can refer to general reference texts for detailed descriptions of known techniques discussed herein or equivalent techniques. These texts include Current Protocols in Molecular Biology, Ausubel, et al., eds., John Wiley & Sons, N.Y. (1989), and supplements through September (1998), Molecular Cloning, A Laboratory Manual, Sambrook et al, 2^{nd} Ed., Cold Spring Harbor Press, Cold Spring Harbor, N.Y. (1989), Genome Analysis: A Laboratory Manual 1: Analyzing DNA, Birren et al., Cold Spring Harbor Press, Cold Spring Harbor, N.Y. (1997); Genome Analysis: A Laboratory Manual 2: Detecting Genes, Birren et al., Cold Spring Harbor Press, Cold Spring Harbor, N.Y. (1998); Genome Analysis: A Laboratory Manual 3: Cloning Systems, Birren et al., Cold Spring Harbor Press, Cold Spring Harbor, N.Y. (1999); Genome Analysis: A Laboratory Manual 4: Mapping Genomes, Birren et al., Cold Spring Harbor Press, Cold Spring Harbor, N.Y. (1999); Plant Molecular Biology: A Laboratory Manual, Clark, Springer-Verlag, Berlin, (1997), Methods in Plant Molecular Biology, Maliga et al., Cold Spring Harbor Press, Cold Spring Harbor, N.Y. (1995), each of which is incorporated herein by reference in its entirety. These texts can, of course, also be referred to in making or using an aspect of the invention.

The gene coding for imidazolinone-resistant ALS may be modified in a variety of ways, truncating either or both of the 5′- or 3′-termini, extending the 5′- or 3′-termini, or modifying codons for amino acid substitution. For instance, the gene may be truncated or extended by as many as 50 codons, usually not more than about 20 codons. Combinations of substitution, truncation and extension may be employed. Thus the gene may be manipulated in a variety of ways to change the characteristic of the protein encoded, for convenience in manipulation of the plasmids, or the like.

The nucleic acid molecules of the present invention comprise at least one of the nucleic acid sequences set forth in SEQ ID NO: 1, SEQ ID NO: 2, and SEQ ID NO: 25 and fragments of either that encodes the amino acid substitutions of the invention. An ALS gene isolated from Arabidopsis thaliana ecotype Columbia with nucleic acid sequence of SEQ ID NO: 1 encodes functional ALS with amino acid sequence of SEQ ID NO: 3 which has the alanine-to threonine substitution at amino acid sequence position 122. An ALS gene isolated from Arabidopsis thaliana ecotype Columbia with nucleic acid sequence of SEQ ID NO: 2 encodes functional ALS with amino acid sequence of SEQ ID NO: 4 which has the alanine-to-valine substitution at amino acid sequence position 205. An ALS gene isolated from Arabidopsis thaliana ecotype Landsberg erecta with nucleic acid sequence of SEQ ID NO: 25 encodes functional ALS with amino acid sequence of SEQ ID NO: 26 which has the alanine-to-valine substitution at amino acid sequence position 205. In another aspect of the present invention, one or more of the nucleic acid molecules of the present invention share at least 60% sequence identity with one or more of the nucleic acid sequences set forth in SEQ ID NO: 1, SEQ ID NO:2 and SEQ ID NO: 25 or complements thereof or fragments of either. In a further aspect of the present invention, one or more of the nucleic acid molecules of the present invention share at least 70% or more, e.g., at least 80%, sequence identity with one or more of the nucleic acid sequences set forth in SEQ ID NO: 1, SEQ ID NO: 2, and SEQ ID NO:25 or complements thereof or fragments of either. In a more preferred aspect of the present invention, one or more of the nucleic acid molecules of the present invention share at least 90% or more, e.g., at least 95% and up to 100% sequence identity with one or more of the nucleic acid sequences set forth in SEQ ID NO: 1, SEQ ID NO: 2, and SEQ ID NO:25 complements thereof or fragments of either. Once the molecular basis of imidazolinone-resistance is known, imidazolinone-sensitive plant ALS genes can be specifically modified to confer imidazolinone-resistance.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e. the entire reference sequence or a smaller defined part of the reference sequence. “Percent identity” is the identity fraction times 100.

Useful methods for determining sequence identity are disclosed in Guide to Huge Computers, Martin J. Bishop, ed., Academic Press, San Diego, 1994, and Carillo, H., and Lipton, D., SIAM J Applied Math (1988) 48:1073, each of which is incorporated herein by reference. More particularly, preferred computer programs for determining sequence identity include the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. Mol. Biol. 215:403-410 (1990), incorporated herein by reference; version 2.0 or higher of BLAST programs Docket No. 38-10(15820)B allows the introduction of gaps (deletions and insertions) into alignments; for peptide sequence BLASTX can be used to determine sequence identity; and, for polynucleotide sequence BLASTN can be used to determine sequence identity.

For purposes of this invention “percent identity” shall be determined using BLASTX version 2.0.08 for translated nucleotide sequences and BLASTN version 2.0.08 for polynucleotide sequences.

(b) Proteins

In a preferred embodiment the present invention provides imidazolinone-resistant functional ALS, e.g. SEQ ID NO: 3, SEQ ID NO: 4, or SEQ ID NO: 26 and homologs thereof. More particularly such homologs will have alanine-to-threonine substitution at amino acid sequence position 122 (SEQ ID NO: 3) or alanine-to-valine substitution at amino acid sequence position 205 (SEQ ID NO: 4 and SEQ ID NO: 26). As used herein, “homolog” means at least 60% sequence identity of the nucleic acid molecule encoding the protein of interest of the same function and at least a lower activity, preferably at least 80% identity in the 30 residues region centered on an amino acid substitution at position 122 or 205 of FIGS. 1 and 2.

In an embodiment of the present invention is a homolog of another plant protein, e.g., cotton, maize, soy, wheat, canola, rice, sunflower or Arabidopsis thaliana. In another preferred embodiment of the present invention, SEQ ID NO: 3 or SEQ ID NO: 4 or SEQ ID NO: 26 of the present invention is a homolog of a viral, bacterial, fungal or animal protein. In a preferred embodiment of the present invention, the nucleic molecule of the present invention encodes a mutant ALS where the protein exhibits a BLAST E value score of less than 1E-8, preferably a BLAST E value score of between about 1E-30 and about 1E-8, even more preferably a BLAST probability E value score of less than 1E-30 with its homolog.

In another further aspect of the present invention, nucleic acid molecules of the present invention can comprise sequences which differ from those encoding a protein or fragment thereof in SEQ ID NO: 1, SEQ ID NO: 2, and SEQ ID NO:25 due to fact that the different nucleic acid sequence encodes a protein having one or more conservative amino acid changes. It is understood that codons capable of coding for such conservative amino acid substitutions are known in the art.

It is well known in the art that one or more amino acids in a native sequence can be substituted with another amino acid(s), the charge and polarity of which are similar to that of the native amino acid, i.e., a conservative amino acid substitution, resulting in a silent change. Conserved substitutions for an amino acid within the native polypeptide sequence can be selected from other members of the class to which the naturally occurring amino acid belongs. Amino acids can be divided into the following four groups: (1) acidic amino acids, (2) basic amino acids, (3) neutral polar amino acids, and (4) neutral nonpolar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral nonpolar (hydrophobic) amino acids such as alanine, leucine, isoleucine, valine, proline, phenylalanine, tryptophan, and methionine.

Conservative amino acid changes within the native polypeptides sequence can be made by substituting one amino acid within one of these groups with another amino acid within the same group. Biologically functional equivalents of the proteins or fragments thereof of the present invention can have ten or fewer conservative amino acid changes, more preferably seven or fewer conservative amino acid changes, and most preferably five or fewer conservative amino acid changes. The encoding nucleotide sequence will thus have corresponding base substitutions, permitting it to encode biologically functional equivalent forms of the proteins or fragments of the present invention.

It is understood that certain amino acids may be substituted for other amino acids in a protein structure without appreciable loss of interactive binding capacity with structures such as, for example, antigen-binding regions of antibodies or binding sites on substrate molecules. Because it is the interactive capacity and nature of a protein that defines that protein's biological functional activity, certain amino acid sequence substitutions can be made in a protein sequence and, of course, its underlying DNA coding sequence and, nevertheless, obtain a protein with like properties. It is thus contemplated by the inventors that various changes may be made in the peptide sequences of the proteins or fragments of the present invention, or corresponding DNA sequences that encode said peptides, without appreciable loss of their biological utility or activity. It is understood that codons capable of coding for such amino acid changes are known in the art.

In making such changes, the hydropathic index of amino acids may be considered. The importance of the hydropathic amino acid index in conferring interactive biological function on a protein is generally understood in the art (Kyte and Doolittle, J. Mol. Biol. 157, 105-132 (1982), incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as govern by the hydrophilicity of its adjacent amino acids, correlates with a biological property of the protein. In a further aspect of the present invention, one or more of the nucleic acid molecules of the present invention differ in nucleic acid sequence from those encoding a peptide set forth in SEQ ID NO: 1, SEQ ID NO: 2, and SEQ ID NO:25 or fragment thereof due to the fact that one or more codons encoding an amino acid has been substituted for a codon that encodes a nonessential substitution of the amino acid originally encoded.

Agents of the invention include nucleic acid molecules that encode at least about a contiguous 10 amino acid region of a protein of the present invention, more preferably at least about a contiguous 11 to 14 or larger amino acid region of a protein of the present invention. In a preferred embodiment the protein is selected from the group consisting of a plant, more preferably a maize, soybean, wheat, cotton, canola, rice, sunflower or Arabidopsis thaliana protein. The present invention provides nucleic acid sequences encoding functional ALS enzymes resistant to imidazolinone herbicides. In one embodiment of this invention, the sequences comprise a mutation in the codon encoding the amino acid alanine at position 122 in the Arabidopsis ALS sequence, or in the corresponding alignment position in other plant sequences as in FIG. 1. In another embodiment of this invention, the sequences comprise a mutation in the codon encoding the amino acid alanine at position 205 in the Arabidopsis ALS sequence, or in the corresponding alignment position in other plant sequences as in FIG. 2. Other plants, such as wheat (a monocot), are also known to exhibit imidazolinone specific mutations (e.g., ATCC Nos. 40994-97). In Arabidopsis thaliana the wild type sequence has an alanine at each position. In alternative embodiments, the substitution of alanine to threonine at position 122 may be a neutral polar amino acid, e.g. serine, cysteine, tyrosine, asparagine, or glutamine, and more preferably an alternate to threonine is serine. In another alternate embodiment, the substitution of alanine to valine at position 205, may be a neutral non-polar amino acid, e.g. leucine, isoleucine, proline, phenylalanine, tryptophan, and methionine, and more preferably leucine or isoleucine. Although the claimed sequences are originally derived from Arabidopsis thaliana, the novel sequences are useful in methods for producing imidazolinone resistant cells in any type of plant, said methods comprising transforming a target plant cell with one or more of the altered sequences provided herein.

(c) Vectors and Constructs

The DNA sequence containing the structural gene expressing the imidazolinone-resistant ALS may be joined to a wide variety of other DNA sequences for introduction into an appropriate host cell. The companion sequence will depend upon the nature of the host, the manner of introduction of the DNA sequence into the host, and whether episomal maintenance or integration is desired.

Whether the DNA may be replicated as an episomal element, or whether the DNA may be integrated into the host genome and the structural gene expressed in the host, will be determined by the presence of a competent replication system in the DNA construction. Episomal elements may be employed, such as tumor inducing plasmids, e.g., Ti or Ri, or fragments thereof, or viruses, e.g., CaMV, TMV or fragments thereof, which are not lethal to the host, and where the structural gene is present in such episomal elements in a manner allowing for expression of the structural gene. Of particular interest are fragments having the replication function and lacking other functions such as oncogenesis, virulence, and the like.

To introduce isolated genes or groups of genes into the genome of plant cells an efficient host gene vector system is necessary. The foreign genes should be expressed in the transformed plant cells and stably transmitted, somatically or sexually to a second generation of cells produced. The vector should be capable of introducing, maintaining, and expressing a gene from a variety of sources in the plant cells. Additionally, it should be possible to introduce the vector into a variety of plants, and at a site permitting effective gene expression. Moreover, to be effective, the selected gene must be passed on to progeny by normal reproduction.

The fragments obtained from the imidazolinone-resistant source may be cloned employing an appropriate cloning vector. Cloning can be carried out in an appropriate unicellular microorganism, e.g., a bacterium, such as E. coli, or Salmonella. In particular, one may use a phage, where partial or complete digestion provides fragments having about the desired size. For example, the phage lambda may be partially digested with an appropriate restriction enzyme and ligated to fragments resulting from either partial or complete digestion of a plasmid, chromosome, or fragment thereof. Packaging will insure that only fragments of the desired size will be packaged and transduced into the host organism.

The host organism may be selected for ALS activity. The recipient strains may be modified to provide for appropriate genetic traits which allow for selection of transductants. In microorganisms, the transductants may be used for conjugation to other microorganisms, using a mobilizing plasmid as required. Various techniques may be used for further reducing the size of the fragment containing the structural gene for the imidazolinone-resistant ALS activity. For example, the phage vector may be isolated, cleaved with a variety of restriction endonucleases, e.g., EcoRI, BamHI, and the like, and the resulting fragments cloned in an appropriate vector, conveniently the phage vector previously used. Instead of a phage vector, a variety of cloning vectors are available of suitable size.

The fragment including flanking regions will be about 11.5 kb. Of particular interest, is a XbaI fragment from Arabidopis thaliana. More particularly the subcloned fragment is about 5.8 kb; specifically the gene is about 2.1 kb. Preferably one skilled in the art can design PCR primers to amplify an imidazolinone-resistant ALS gene.

The imidazolinone-resistant ALS enzyme may be expressed by any convenient source, either prokaryotic or eukaryotic, including bacteria, yeast, filamentous fungus, plant cells, etc. Where secretion is not obtained, the enzyme may be isolated by lysing the cells and isolating the mutant ALS according to known ways. Useful ways include chromatography, electrophoresis, affinity chromatography, and the like.

The DNA sequence encoding for the imidazolinone-resistant ALS activity may be used in a variety of ways. The DNA sequence may be used as a probe for the isolation of mutated or wild type ALS sequences. Also saturation or site-directed mutagenesis could be performed on a plant ALS gene to select for mutants expressing greater levels of herbicide-resistance, as well as resistance to more classes of herbicide. Alternatively, the DNA sequence may be used for integration by recombination into a host to provide imidazolinone resistance in the host. The mutant ALS gene can also be used as selection marker in the plant transformation experiments using the imidazolinone herbicide as the selection agent.

A vector or construct may also include a selectable marker. Selectable markers may also be used to select for plants or plant cells that contain the exogenous genetic material. Examples of such include, but are not limited to: a neomycin phosphotransferase gene (U.S. Pat. No. 5,034,322, incorporated herein by reference), which codes for kanamycin resistance and can be selected for using kanamycin, G418, etc.; a bar gene which codes for bialaphos resistance; genes which encode glyphosate resistance (U.S. Pat. Nos. 4,940,835; 5,188,642; 4,971,908; 5,627,061, each of which is incorporated herein by reference); a nitrilase gene which confers resistance to bromoxynil (Stalker et al., J. Biol. Chem. 263:6310-6314 (1988), incorporated herein by reference); a mutant acetolactate synthase gene (ALS) which confers imidazolinone or sulphonylurea resistance (European Patent Application 154,204 (Sep. 11, 1985), incorporated herein by reference); and a methotrexate resistant DHFR gene (Thillet et al., J. Biol. Chem. 263:12500-12508 (1988), incorporated herein by reference). Mutant ALS genes of this invention may be used as a selectable marker on a vector used in transformation experiments in which ALS is not the gene of interest.

(d) Transformation

With plant cells, the structural gene as part of a construction may be introduced into a plant cell nucleus by a variety of genetic transformation methods but preferably by Agrobacterium mediated transformation, gene-gun or particle bombardment or micropipette injection for integration by recombination into the host genome. Methods for the genetic transformation of plants are known to those of skill in the art. For example, methods which have been described for the genetic transformation of plants include electroporation (U.S. Pat. No. 5,384,253), electrotransformation (U.S. Pat. No. 5,371,003), microprojectile bombardment (U.S. Pat. No. 5,550,318; U.S. Pat. No. 5,736,369, U.S. Pat. No. 5,538,880; and PCT Publication WO 95/06128), Agrobacterium-mediated transformation (Horsch et al., Science 227:1229 (1985); U.S. Pat. No. 5,591,616 and EP Publication EP672752), direct DNA uptake transformation of protoplasts (Omirulleh et al., 1993) and silicon carbide fiber-mediated transformation (U.S. Pat. No. 5,302,532 and U.S. Pat. No. 5,464,765).

Although Agrobacterium tumefaciens effectively transform only dicots, the Ti plasmid permits the efficacious manipulation of the bacteria to act as vectors in monocotyledonous crop plants, i.e., wheat, barley, rice, rye, etc. Alternatively, Ti plasmids or other plasmids may be introduced into the monocots by artificial methods such as microinjection, or fusion between the monocot protoplasts and bacterial spheroplasts containing the T-region which could then be integrated into the plant nuclear DNA.

By employing the T-DNA right border, or both borders, where the borders flank an expression cassette comprising the imidazolinone-resistant ALS structural gene under transcriptional and translational regulatory signals for initiation and termination recognized by the plant host, the expression cassette may be integrated into the plant genome and provide for expression of the imidazolinone-resistant ALS enzyme in the plant cell at various stages of differentiation. Various constructs can be prepared providing for expression in plant cells.

To provide for transcription, a variety of transcriptional initiation regions (promoter regions), either constitutive or inducible, may be employed. The transcriptional initiation region is joined to the structural gene encoding the imidazolinone-resistant ALS activity to provide for transcriptional initiation upstream from the initiation codon, normally within about 200 bases of the initiation codon, where the untranslated 5′-region lacks an ATG. The 3′-end of the structural gene will have one or more stop codons which will be joined to a transcriptional termination region functional in a plant host, which termination region may be associated with the same or different structural gene as the initiation region.

The expression cassette is characterized by having the initiation region, the structural gene under the transcriptional control of the initiation region, and the termination region providing for termination of transcription and processing of the messenger RNA, in the direction of transcription as appropriate.

Transcriptional and translational regulatory regions, conveniently tml promoter and terminator regions from A. tumefaciens may be employed, which allow for constitutive expression of the imidazolinone-resistant ALS gene. Alternatively, other promoters and/or terminators may be employed, particularly promoters which provide for inducible expression or regulated expression in a plant host. Promoter regions which may be used from the Ti-plasmid include opine promoters, such as the octopine synthase promoter, nopaline synthase promoter, agropine synthase promoter, mannopine synthase promoter, or the like. Other promoters include viral promoters, such as CaMV Region VI promoter or full length (35S) promoter, the promoters associated with the ribulose-1,5-bisphosphate carboxylase genes, e.g., the small subunit, genes associated with phaseolin, protein storage, B-conglycinin, cellulose formation, or the like.

The various sequences may be joined together in conventional ways. The promoter region may be identified by the region being 5′ from the structural gene, for example, the tml gene, and may be selected and isolated by restriction mapping and sequencing. Similarly, the terminator region may be isolated as the region 3′ from the structural gene. The sequences may be cloned and joined in the proper orientation to provide for constitutive expression of the imidazolinone-resistant ALS gene in a plant host.

The expression cassette expressing the imidazolinone-resistant ALS enzyme may be introduced into a wide variety of plants, both monocotyledon and dicotyledon, including maize, wheat, soybean, tobacco, cotton, tomatoes, potatoes, Brassica species, rice, peanuts, petunia, sunflower, sugar beet, turfgrass, etc. The gene may be present in cells or plant parts including callus, tissue, roots, tubers, propagules, plantlets, seeds leaves, seedlings, pollen, or the like.

By providing for imidazolinone-resistant plants, a variety of imidazolinone herbicides may be employed for protecting crops from weeds, so as to enhance crop growth and reduce competition for nutrients. The mutant ALS gene can be introduced into plants, preferably crop plants, and regenerated to produce a new family of transgenic plants which possess increased resistance to imidazolinone as compared with that possessed by the corresponding wild plants. An imidazolinone, such as imazapyr, could be used by itself for post emergence control of weeds with transgenically protected crops, such as sunflower, soybeans, corn, cotton, canola, wheat, rice etc., or alternatively, in combination formulations with other products.

Having now generally described the invention, the same will be more readily understood through reference to the following examples, which specifically define preferred techniques for the production of an imidazolinone herbicide resistant Arabidopsis thaliana, sequencing to determine the mutation in its ALS gene, and a process for conferring imidazolinone herbicide resistance to plants other than Arabidopsis thaliana.

The following examples are offered by way of illustration and are not intended to be limiting of the present invention.

### EXAMPLE 1

This example serves to illustrate isolation of mutant Arabidopsis thaliana lines resistant to an imidazolinone herbicide.

A. Primary Screen

EMS-mutagenized M₂ generation Arabidopsis thaliana (cv. Col-0) seeds were obtained from Lehle Seeds, Round Rock, Texas under catalog number M2E02-02. Flats were seeded to provide approximately one million ems-mutagenized seeds. The seeded flats were then watered by sub-irrigation, covered with transparent plastic domes, and maintained at 4° C. and 70% relative humidity for 4 days in the dark. At the end of this period the growth chamber conditions were reset to 21° C., 70% relative humidity under a 16 hour photoperiod, with soil-surface light intensity adjusted to approximately 150 uEinsteins/m²/s. The flats were maintained under these conditions for seven days prior to imazethapyr, an imidazolinone treatments. The plastic domes were removed 2 days before the treatments to acclimate the plants to ambient humidity.

B. Imidazolinone Treatments

Based on repeated dose-response experiments using 7-day-old Arabidopsis seedlings grown under the conditions described above, it was determined that the I₁₀₀ for imazethapyr was approximately 0.035 lbs. active ingredient per acre (a.i./acre). EMS-mutagenized seedlings were screened at a rate equivalent to approximately twice the 1100 value, or 0.07 lbs. a.i./acre. Fresh imazethapyr treatment solutions were prepared by dissolving 220 mg of active ingredient in 1.0 ml DMSO, which was then added drop wise to 1.0 liter of solution containing tween-20 at 0.1% (v/v) while stirring. Applications were made using an automated cabinet sprayer equipped with a moving boom. Solutions were applied using a 8002E TeeJet tip at a boom height of 16 inches, delivering a diluent volume of 38.5 gal/acre at a pressure of 28 p.s.i. Seedlings were then returned to the growth chamber, and scored for foliar damage at 16 days post-treatment.

C. Isolation and Confirmation of Mutants

From a primary screen of approximately one million 7-day-old M₂ seedlings using the protocol described above, five candidate surviving seedlings were identified. To confirm true resistance, each of the surviving plants were allowed to self-pollinate and approximately 150 M₃ progeny seedlings were grown substantially as described for the primary screens. In addition, a control experiment was conducted in parallel with wild-type seedlings to demonstrate that the reduced seeding density did not impact the 1100 values. The M₃ lines re-tested in this manner were confirmed as being resistant by observing differences between mutant plants and wild type plants in the presence of an imidazolinone herbicide.

### EXAMPLE 2

This example serves to illustrate methods of DNA preparation and sequence determination of the gene encoding mutant ALS in Arabidopsis thaliana.

Genomic DNA of imidazolinone-resistant mutants isolated from Arabidopsis thaliana ecotype Columbia and is prepared by any of several standard methods. For instance:

1. A single leaf from the plant whose DNA is to be sequenced is placed in a microcentrifuge tube.

2. The leaf is lyophilized for 24 hours.

3. The leaf is ground to a fine powder in the tube by vortexing with a steel bearing.

4. 350 microliters extraction buffer (200 mM Tris pH 7.5, 250 mM NaCl, 25 mM EDTA, 0.5% SDS is added).

5. Tube is placed at 65 C for 60 minutes.

6. 100 microliters of 5 M potassium acetate pH 7.5-8.0 is added.

7. Tube is centrifuged to precipitate plant debris (3000 RPM, 15 minutes)

8. 220 of ice-cold isopropanol is added.

9. Tube is centrifuged to precipitate DNA (3000 RPM, 15 minutes)

10. Pellet is washed with 210 microliters 75% ethanol.

11. Ethanol is removed and pellet is allowed to dry overnight.

12. 400 microliters 10 mM Tris pH 8.0, 0.05 M EDTA is added.

13. Tube is place at 65 C for ten minutes to dissolve the pellet

Aliquots of the genomic DNA solution described above are used to amplify the ALS gene by PCR. Since the sequence of the ALS gene(base pairs 37085-39097 on BAC T8P19, GI:6523080) and flanking regions is known for Arabidopsis thaliana ecotype Columbia, DNA primers are designed to specifically amplify just the ALS gene and a small amount of flanking DNA from each mutant. Pairs of primers designed to amplify overlapping pieces of ALS DNA roughly 500 base pairs long are shown in Table A.

TABLE A

| Primer Pair | Approximate ALS | | |
|---|---|---|---|
| Number | gene coordinates | Forward primer | Reverse primer |
| 1 | 0001-0500 | SEQ ID NO: 5 | SEQ ID NO: 6 |
| 2 | 0251-0750 | SEQ ID NO: 7 | SEQ ID NO: 8 |
| 3 | 0501-1000 | SEQ ID NO: 9 | SEQ ID NO: 10 |
| 4 | 0751-1250 | SEQ ID NO: 11 | SEQ ID NO: 12 |
| 5 | 1001-1500 | SEQ ID NO: 13 | SEQ ID NO: 14 |
| 6 | 1251-1750 | SEQ ID NO: 15 | SEQ ID NO: 16 |
| 7 | 1501-2000 | SEQ ID NO: 17 | SEQ ID NO: 18 |
| 8 | 1751-2250 | SEQ ID NO: 19 | SEQ ID NO: 20 |
| 9 | 2001-2500 | SEQ ID NO: 21 | SEQ ID NO: 22 |

The amplified DNA fragments are purified and sequenced by a variety of methods. The same primers used to amplify DNA fragments are used to sequence those fragments.

Two basic methods can be used for DNA sequencing, the chain termination method of Sanger et al., Proc. Natl. Acad. Sci. (U.S.A.) 74:5463-5467 (1977), the entirety of which is herein incorporated by reference and the chemical degradation method of Maxam and Gilbert, Proc. Natl. Acad. Sci. (U.S.A.) 74:560-564 (1977), the entirety of which is herein incorporated by reference. Automation and advances in technology such as the replacement of radioisotopes with fluorescence-based sequencing have reduced the effort required to sequence DNA (Craxton, Methods 2:20-26 (1991), the entirety of which is herein incorporated by reference; Ju et al., Proc. Natl. Acad. Sci. (U.S.A.) 92:4347-4351 (1995), the entirety of which is herein incorporated by reference; Tabor and Richardson, Proc. Natl. Acad. Sci. (U.S.A.) 92:6339-6343 (1995), the entirety of which is herein incorporated by reference). Automated sequencers are available from, for example, Pharmacia Biotech, Inc., Piscataway, N.J. (Pharmacia ALF), LI-COR, Inc., Lincoln, Nebr. (LI-COR 4,000) and Millipore, Bedford, Mass. (Millipore BaseStation).

In addition, advances in capillary gel electrophoresis have also reduced the effort required to sequence DNA and such advances provide a rapid high resolution approach for sequencing DNA samples (Swerdlow and Gesteland, Nucleic Acids Res. 18:1415-1419 (1990); Smith, Nature 349:812-813 (1991); Luckey et al., Methods Enzymol. 218:154-172 (1993); Lu et al., J. Chromatog. A. 680:497-501 (1994); Carson et al., Anal. Chem. 65:3219-3226 (1993); Huang et al., Anal. Chem. 64:2149-2154 (1992); Kheterpal et al., Electrophoresis 17:1852-1859 (1996); Quesada and Zhang, Electrophoresis 17:1841-1851 (1996); Baba, Yakugaku Zasshi 117:265-281 (1997), all of which are herein incorporated by reference in their entirety).

A number of sequencing techniques are known in the art, including fluorescence-based sequencing methodologies. These methods have the detection, automation and instrumentation capability necessary for the analysis of large volumes of sequence data. Currently, the 377 DNA Sequencer (Perkin-Elmer Corp., Applied Biosystems Div., Foster City, Calif.) allows the most rapid electrophoresis and data collection. With these types of automated systems, fluorescent dye-labeled sequence reaction products are detected and data entered directly into the computer, producing a chromatogram that is subsequently viewed, stored, and analyzed using the corresponding software programs. These methods are known to those of skill in the art and have been described and reviewed (Birren et al., Genome Analysis: Analyzing DNA,1, Cold Spring Harbor, N.Y., the entirety of which is herein incorporated by reference).

PHRED (available from the University of Washington Genome Center) is used to call the bases from the sequence trace files. PHRED uses Fourier methods to examine the four base traces in the region surrounding each point in the data set in order to predict a series of evenly spaced predicted locations. That is, it determines where the peaks would be centered if there were no compressions, dropouts, or other factors shifting the peaks from their “true” locations. Next, PHRED examines each trace to find the centers of the actual, or observed peaks and the areas of these peaks relative to their neighbors. The peaks are detected independently along each of the four traces so many peaks overlap. A dynamic programming algorithm is used to match the observed peaks detected in the second step with the predicted peak locations found in the first step.

Once the sequence of the ALS gene of imidazolinone-resistant mutants has been determined, it is compared to the known sequence of the wild type gene. One imidazolinone-resistant mutant was determined to have an ALS gene with nucleic acid sequence of SEQ ID NO: 1 which encodes ALS with an alanine to threonine substitution at amino acid position 122, with reference to the Arabidopsis ALS, as shown in amino acid sequence of SEQ ID NO: 3. Another imidazolinone-resistant mutant was determined to have an ALS gene with nucleic acid sequence of SEQ ID NO: 2 which encodes ALS with a alanine to valine substitution at amino acid position 205, with reference to the Arabidopsis ALS, as shown in amino acid sequence of SEQ ID NO: 4. Moreover, the amino acid sequence of the wild type ALS from other organisms can be aligned to the amino acid sequence of Arabidopsis wild type ALS at the region of the 122 and 205 substitutions to allow for design of imidazolinone resistant ALS in those organisms.

In FIG. 1 amino acid sequence from ALS from Brassica napus (found at GenBank under GI:17771), Gossypium hirsutum (GI:1130681), Nicotiana tabacum (GI:19776), Glycine max (from a proprietary cDNA library) and Zea mays (GI:22138) was aligned to the 31 amino acid region centered at position 122 of Arabidopsis thaliana ALS. In FIG. 2, the amino acid sequence of ALS from the same organisms is aligned to the 31 amino acid sequence region centered at position 205 of Arabidopsis thaliana ALS. Sequence determination of the wild type ALS protein of each Arabidopsis thaliana ecotype analyzed (Columbia and Landsberg erecta) shows that they are identical in each of the 31 amino acid regions shown in FIGS. 1 and 2 (SEQ ID NO: 25 and SEQ ID NO: 31, respectively).

### EXAMPLE 3

This example serves to illustrate construction of a T-DNA vector containing a mutant Arabidopsis ALS gene of this invention. The sequence of wild type Arabidopsis thaliana ALS is known (base pairs 37085-39097 on BAC T8P19, GI:6523080). Based on this sequence it is possible to design primers and amplify the entire gene from the mutant versions of the gene (Ala122Thr and Ala205Val) using the polymerase chain reaction.

The plasmid pCGN8640 is a T-DNA vector that can be used to clone exogenous genes and transfer them into plants using Agrobacterium-mediated transformation. pCGN8640 has the restriction sites BamH1, Not1, HindIII, PstI, and SacI in between the 35S promoter and a transcription terminator. Flanking this DNA are the left border and right border sequences necessary for Agrobacterium transformation. The plasmid also has origins of replication for maintaining the plasmid in both E. coli and Agrobacterium tumefaciens strain ABI. A spectinomycin resistance gene on the plasmid can be used to select for the presence of the plasmid in both E. coli and Agrobacterium tumefaciens. An ALS gene is prepared by PCR for insertion into the T-DNA vector. Two primers useful for amplifying an ALS gene from mutant lines of Arabidopsis are given in SEQ ID NO: 23 (forward primer) and SEQ ID NO: 24 (reverse primer). The primer described by SEQ ID NO: 23 has DNA sequence complementary to the 5′ end of ALS and also contains a NotI restriction site. The primer described by SEQ ID NO: 24 has DNA sequence complementary to the 3′ end of ALS and also contains a PstI restriction site. Mutant ALS genes are amplified by PCR techniques using these two primers. Both the amplified DNA and the pCGN8640 vector are cut with the restriction enzymes NotI and PstI. The resulting fragments are gel-purified, ligated together, and transformed into E. coli.

Plasmid DNA (pCGN8640 with the inserted mutant ALS gene) is isolated from E. coli by selecting for spectinomycin resistance (100 ug/ml), plasmid DNA is isolated, and the presence to the desired insert in pCGN8640 is verified by digestion with NotI and PstI. Undigested plasmid is transformed into Agrobacterium tumefaciens by selection for spectinomycin resistance (100 ug/ml).

### EXAMPLE 4

This example serves to illustrate transformation of mutant ALS into a host cell. Agrobacterium tumefaciens carrying the plasmid construct is used to transform Arabidopsis using the dipping method (Clough S J and Bent AF. Plant J. December 1998;16(6):735-43). An ALS gene having SEQ ID NO: 1 or SEQ ID NO: 2 is amplified by PCR and cloned into a T-DNA expression vector such as pCGN8640. The resulting construct is transformed into Agrobacterium tumefaciens by electroporation (Current Protocols in Molecular Biology, Ausubel et al., eds., Hohn Wiley & Sons, N.Y., 1989). A one-liter culture of Agrobacterium tumefaciens containing the T-DNA construct is grown up. The culture is sedimented by centrifugation and the bacterial cells are resuspended in 2 liters of water with 5% sucrose and 0.02% Silwet L-77 surfactant (OSI Specialties, Inc., Danbury, Conn., USA). Flowering plants of Arabidopsis thaliana are dipped into this bacterial solution such that the inflorescences are submerged. While submerged in the bacterial solution, the plants are gently agitated for about 20 seconds. After dipping, plants are covered with a plastic dome and are placed in a lighted greenhouse or growth chamber for 24 hours. The plastic dome is removed after 24 hours and the plants are not watered until the soil is dry (3 to 7 days). After this time period, a normal watering schedule is resumed. Approximately 1 month after dipping, seeds are harvested from the plants. Seeds are imbibed in water, placed at 4 degrees C. for 1 to 4 days, and then planted in soil. Four days after seedling emergence plants are sprayed with imazapyr or imazethapyr (imidazolinone herbicides) at twice the L100 concentration (0.07 lbs active ingredient/acre). Those plants which are transformed with the mutant version of the ALS gene (SEQ ID NO: 1 or SEQ ID NO: 2) will survive and those which are not will die. If Arabidopsis thaliana ecotype Columbia is used for the transformation, then the frequency of resistant transformants will be approximately 1%.

### REFERENCES

Each document and patent cited or identified herein, whether it is specifically incorporated by reference or not, is hereby incorporated herein by reference in its entirety. In addition, these references, as well as each of those cited can be relied upon to make and use aspects of the invention.

What we claim is:

1. A nucleic acid molecule encoding functional ALS which has (a) an alanine-to-threonine substitution at amino acid sequence position 122, or (b) an alanine-to-valine substitution at amino acid sequence position 205, relative to the amino acid sequence alignment of FIGS. 1 and 2.

2. A transformation vector comprising the nucleic acid molecule of claim 1.

3. A host cell comprising the nucleic acid molecule of claim 1.

4. A host cell of claim 3 which is a plant cell or a bacterial cell.

5. A host cell of claim 4 which is an imidazolinone-resistant plant selected from the group consisting of Arabidopsis thaliana, maize, soybean, wheat, cotton, canola, rice and sunflower.

6. A host cell of claim 4 which is recombinant.

7. A transformed plant exhibiting imidazolinone resistance having a nucleic acid molecule which comprises: (a) an exogenous promoter region which functions in a plant cell to cause the production of a mRNA molecule; (b) a structural nucleic acid molecule encoding functional ALS comprising an amino acid sequence of SEQ ID NO: 3 or SEQ ID NO: 4 or SEQ ID NO: 26 or a homolog thereof having an alanine-to-threonine substitution at position 122 or an alanine-to-valine substitution at position 205, and (c) a 3′ non-translated sequence that functions in the plant cell to cause termination of transcription and addition of polyadenylated ribonucleotides to a 3′ end of the mRNA molecule.

8. The transformed plant according to claim 7, wherein said plant is selected from the group of rice, cotton, wheat, canola, maize, soybean and Arabidopsis thaliana.

9. The transformed plant of claim 8 wherein the structural nucleic acid molecule has a nucleic acid sequence of SEQ ID NO: 1 or SEQ ID NO:2 or SEQ ID NO: 25 or homologs thereof which encode an ALS with an alanine-to-threonine substitution at position 122 or an alanine-to-valine substitution at position 205.

10. A method of conferring imidazolinone-specific resistance to a plant cell which comprises providing the plant cell with the nucleic acid sequence of claim 1.

11. A nucleic acid construct comprising the sequence of claim 1 linked to a gene encoding an agronomically useful trait.

12. A method of conferring imidazolinone resistance to a plant comprising providing said plant with a nucleic acid molecule of claim 1.

13. A method for determining the imidazolinone tolerance of a plant comprising detecting the presence of a nucleic acid molecule of claim 1.

14. A method for introgressing an agronomically useful trait into a plant comprising: (a) constructing a vector comprising SEQ ID NO: 1 or SEQ ID NO: 2 or SEQ ID NO: 25 or homologs thereof which encode an alanine-to-threonine substitution at position 122 or an alanine-to-valine substitution at position 205, operably linked to a gene for said agronomically useful trait; (b) transforming said vector into plant cells; (c) growing plant; and (d) testing plant for introgression of said trait by selecting plants with imidazolinone resistance.

15. A method using imidazolinone resistance as a selectable marker in a cell or organism wherein said resistance is provided by nucleic acid molecule of claim 1.

16. A set of primer pairs for amplifying an ALS gene or fragment thereof comprising at least two oligonucleotides selected from the group consisting of SEQ ID NO: 5 through SEQ ID NO: 24.
